(19) United States
(12) Patent Application Publication
(10) Pub. No.: US 2020/0217801 A1
(43) Pub. Date: Jul. 9, 2020

(54) ELECTROCHEMILUMNISCENCE IMMUNOSENSOR FOR DETECTING HAPTOGLOBIN

(71) Applicant: UNIVERSITI BRUNEI DARUSSALAM, Gadong (BN)

(72) Inventors: Minhaz Uddin AHMED, Brunei (BN); Mohammad RIZWAN, Gadong (BN)

(21) Appl. No.: 16/819,961

(22) Filed: Mar. 16, 2020

(30) Foreign Application Priority Data
Mar. 18, 2019 (BN) BN/N/2019/0031

(51) Int. Cl. G01N 21/76; G01N 33/68; C09K 11/07

(52) U.S. Cl. CPC G01N 21/76; C09K 2211/185; C09K 11/07; G01N 33/6803

(57) ABSTRACT

The present invention discloses an electrochemical immunosensor (ECL). The ECL is configured for detecting Haptoglobin in biological samples. The immunosensor includes nanocomposite of gold nanoparticles, single-walled carbon nanotubes, quantum dots, and chitosan. The immunosensor can also be used for the ECL based detection of other biomarkers and biomolecules, such as Immunoglobulin A and dopamine.

### RELATED APPLICATION

This application claims the benefit of Brunei Patent Application No. filed on Mar. 18, 2019 and entitled “An electrochemilumniscence immunosensor for detecting haptoglobin”, the content of which is incorporated in its entirety herein by reference.

### FIELD OF INVENTION

The present invention relates to an immunosensor. More particularly, the present invention relates to an electrochemiluminescence immunosensor (ECL) for detecting

Haptoglobin in biological samples and methods for fabricating thereof.

### BACKGROUND OF THE INVENTION

Haptoglobin (Hp) is a serum α2-glycoprotein of approximately 100 kDa. It exists as a tetramer, comprising two smaller identical α-chains and two larger identical β-chains. The α-chains are linked to each other by a disulphide bond, and each β-chain is similarly linked to an α-chain. Hp plays an important part in binding and transporting of hemoglobin. However, the plasma concentration of Hp increases several folds in carcinoma, tissue necrosis, coronary artery, schizophrenia and in the event of an inflammatory stimulus such as infection, injury or malignancy, whether local (vascular) or systemic (extravascular). Hp may also be involved in modulating the immune response, autoimmune diseases, and significant inflammatory disorders. Elevated Hp levels are sometimes found in other diseases such as diabetes mellitus, renal disease, and endocrine imbalance. However, there may be a low amount of Hp in plasma in some diseases such as intravascular hemolysis, anemia, malaria, liver disease, jaundice, cirrhosis, mononucleosis and transfusion of incompatible blood.

Therefore, Hp can be a useful biomarker in the diagnostic and monitoring response of the various diseases. Hence, monitoring the rise and fall in the Hp concentration is an essential step for effective treatment, controlling monitoring and screening disease recurrence. However, there is currently no low cost, highly sensitive immunoassay, device or immunosensor available for use in rapid point-of-care detection and quantification of Hp in serum.

Heretofore, laboratory methods to detect Hp may include ELISA (Marsden and Simmonds 1988), competitive immunoassay (Mcnair et al., 19197), radioimmunoassay (Schrijver et al., 1984), affinity matrix (Stöllner et al., 2002), affinity chromatography (Yuch et al., 2007), Westernblotting (Ouyang et al., 1998). Despite the respective advantages of each method, neither of them are straightforward, nor can they be fabricated into highly sensitive devices for mass production or stored as “ready-to-use” devices for an extended period. Therefore, recent efforts have been made to overcome the existing limitation of the immunoassay by fabrication of various types of immunosensors for the detection of the Hp. For example, Wang et al. used magnetic nanobeads, and capillary electrophoresis technique for the fabrication of the Hp-immunosensor based on laser-induced fluorescence detection of the Hp and achieved the low LOD 200 μg mL⁻¹. In another example, Klauke et al. fabricated surface acoustic wave based immunosensor for the detection of the Hp and achieved the low LOD 20 ng mL⁻¹ (Klauke et al. 2013) and Abadieh et al. used Rhodamine and shell of cadmium telluride quantum dots (CdTe-QDs) to fabricate the Hp-immunosensor and achieved the low LOD 2 ng mL⁻¹ (Abadieh et al., 2015). However, methods described above may have certain disadvantages such as a potentially dangerous reagent, radiation hazards, qualified personnel, sophisticated instrumentation, expensive devices, complicated operation process, no storage duration, less sensitivity, and lower selectivity. Such disadvantages significantly limit their practical application, especially for in-situ and routine analysis. Hence, alternative approaches may be required.

Currently, electrochemiluminescence (ECL) has become an essential and powerful analytical technology in many fields. As a result, ECL based immunosensors have gained immense popularity for the fabrication of label free-highly sensitive immunosensors. It is routinely employed due to its high sensitivity and versatility, low background signal, simple optical setup and provide a scientist with excellent temporal and spatial control (Roy et al., 2016). Therefore, various luminophore-coreactant pairs have been used as a source of ECL signal on different electrode materials, such as label-free ECL immunosensor for the detection of transferrin on a luminol reduced gold nanoparticle-modified screen-printed carbon electrode in the presence of luminol H₂O₂ (Kong et al., 2014). In another instance, ECL biosensor for the detection of Staphylococcus on carboxyl graphene/porcine IgG composite deposited on glassy carbon electrode with luminol-H₂O₂ (Yue et al, 2016), luminol-H₂O₂ for the ECL based detection of the glucose, and glucose oxidase activity on the stainless-steel electrode (Kitte et al., 2017) as well as glycated albumin in human serum albumin on the screen-printed carbon electrode (Inoue et al., 2017) and tris(2,2′-bipyridyl)-ruthenium(II) ([Ru(bpy)₃]²⁺)-tri-n-propylamine (TPrA) for the detection of the β2M on CdSe-QDs electrode modified with gold nanoparticles (AuNPs) doped with carbon nano-onions, chitosan (CS) nanocomposite (Rizwan et al., 2017) and nucleic acids detection on the screen-printed carbon electrode (Roy et al., 2016).

There are few patents which disclose using ECL immunosensor technology for the detection of biomarkers.

For example, European patent publication EP2947459A1 discloses a method for determining the concentration of a protein in a gastrointestinal (GI) tract sample taken from a human or an animal. The method employs many technologies; one of them utilized herein is electrochemiluminescence.

Another Chinese patent publication CN102749452B discloses a near-infrared light emission electrochemiluminescence immunoassay, luminescent immunoassay belonging to the technical field of electrochemiluminescence detection; comprising the steps of (1) a near-infrared quantum dots connected with the secondary antibody, the step (2) and a sandwich immunoreactivity prepared ECL immunosensor step (3) electroluminescence chemiluminescence immunodetection immunosensor three steps. The method discloses ECL immunosensor fabricated based on CdTe-QDs and gold electrode.

Another Chinese patent publication CN101706498B discloses ECL immunosensor fabricated based on tris (2,2′-bipyridyl)-ruthenium(II) and a gold electrode. The patent relates to nanometer immune markers, directed by the light-emitting substance of SiO₂ packing (Ru (bpy)³²⁺) and the second antibody (Ab₂) was modified signal having a low concentration of the antigen used for detection of amplification electrochemiluminescence immunoassay method of preparing a light emitting sensor. SiO₂ core-shell nanostructures, not only to maintain a proper chemical nature of the contents but also can effectively prevent the leakage of contents. And because a SiO₂ pellets molecules may be wrapped more markers, so that the detection sensitivity is much improved. The synthesized SiO₂@Ru uniform particle size and good monodispersity, so that each SiO₂@Ru nanoglobules having the same amount of alpha-fetoprotein antibody fixed to improve the reproducibility of the detection. In 0.01-20 ng mL⁻¹ range, the ECL signal value AFP concentration showed an excellent linear relationship, and the detection limit reached 35 pg mL⁻¹.

Another conventional patent publication CN104764737B discloses a monochrome ECL immunodetection method based on quantum dots green radiation, including (1) Preparation of CdSe quantum dot labeled secondary antibody (CdSe QDs-Ab2), and (2) of CdSe quantum dots to marker preparation monochrome ECL immunosensor and (3) drawing working curve, three monochrome ECL immunodetection step. The method discloses high detection sensitivity, detection limit 0.1 fg mL⁻¹, single molecule detection can be achieved for the antigen; high selectivity.

However, there has been no study performed on label-free ECL immunosensor for the sensitive and selective detection of Hp. Therefore, there requires a need for developing ECL based immunosensor for detecting and quantifying Hp. The immunosensor may have necessary features such as including but are not limited to highly sensitive, highly selective, stable, interference-resistant, rapid, label-free, potential to mass-production, ability to detect a wide range of Hp concentration in serum, reliable, eco-friendly and economical.

### SUMMARY OF THE INVENTION

In an aspect, the present invention discloses an electrochemiluminescence immunosensor (ECL). The immunosensor is configured to detect Haptoglobin in biological samples. The immunosensor includes nanocomposite of gold nanoparticles, single-walled carbon nanotubes, quantum dots, and chitosan.

### BRIEF DESCRIPTION OF DRAWINGS

Other objects, features, and advantages of the invention will be apparent from the following description when read concerning the accompanying drawings. In the drawings, wherein like reference numerals denote corresponding parts throughout the several views:

FIG. 1A shows raw materials involved in the preparation of CdTe-QDs/AuNPs/SWCNTs/CS based ECL; FIG. 1B illustrates ECL of bare CNFs-SPE and CdTe-QDs/AuNPs/SWCNTs/CS-nanocomposite modified CNFs-SPE; and FIG. 1C illustrates fabrication of the present ECL immunosensor, by the illustrative embodiment of the present invention

FIG. 2A shows absorption spectra of the CdTe-QDs, AuNPs, CS and CdTe-QDs/AuNPs/SWCNTs/CS;

FIG. 2B shows ECL intensity curve of the (a) bare CNFs-SPE and (b) CdTe-QDs/AuNPs/SWCNTs/CS/CNFs-SPE;

FIG. 2C shows ECL intensity peak bar diagram of the (a) bare CNFs-SPE and (b) CdTe-QDs/AuNPs/SWCNTs/CS/CNFs-SPE;

FIG. 2D shows ECL intensity peak bar diagram of the bare CNFs-SPE and modified CNFs-SPEs;

FIG. 2E shows CV curve of (a) bare CNFs-SPE and (b) CdTe-QDs/AuNPs/SWCNTs/CS/CNFs-SPE;

FIG. 2F shows Nyquist plots of impedance spectra for (a) bare CNFs-SPE and (b) CdTe-QDs/AuNPs/SWCNTs/CS/CNFs-SPE, by the illustrative embodiment of the present invention.

FIG. 3A shows electrochemical, microscopic characterization of the layer-by-layer ECL Hp-immunosensor fabrication, detection of Hp and dose response ECL curve: (a) CNFs-SPEs modified with AuNPs/CdTe-QDs/SWCNTs/CS-nanocomposite spiked with anti-Hp protected with BSA for nonspecific binding, (b) CNFs-SPEs modified with AuNPs/CdTe-QDs/SWCNTs/CS-nanocomposite spiked with anti-Hp, (c) Bare CNFs-SPEs and (d) CNFs-SPEs modified with AuNPs/CdTe-QDs/SWCNTs/CS-nanocomposite;

FIG. 3B shows Electrochemical layer-by-layer study using CV: (a) Bare CNFs-SPEs, (b) CNFs-SPEs modified with AuNPs/CdTe-QDs/SWCNTs/CS-nanocomposite, (c) CNFs-SPEs modified with AuNPs/CdTe-QDs/SWCNTs/CS-nanocomposite spiked with anti-Hp, and (d) CNFs-SPEs modified with AuNPs/CdTe-QDs/SWCNTs/CS-nanocomposite spiked with anti-Hp protected with BSA for nonspecific binding, performed using Fe(CN)₆]^{3/4}-redox probe solution in 0.1 M KCl from −0.4 to 0.7 V with a scan rate of 100 mVs⁻¹;

FIG. 3C shows Microscopic layer-by-layer study: (a) Bare CNF-SPEs, (b) CdTe-QDs/AuNPs/SWCNTs/CS-nanocomposite, (c) CdTeQDs/AuNPs/SWCNTs/CS-nanocomposite spiked with Anti-Hp and (d) CdTeQDs/AuNPs/SWCNTs/CS-nanocomposite spiked with anti-Hp and blocked with BSA, recorded at 5.0 kV, resolution 100 nm and magnification 30 000× in SEI mode;

FIG. 3D shows the ECL intensity curve of immunosensors without Hp and with Hp;

FIG. 3E shows Bar diagram of the ECL intensity peak without Hp and with Hp; and

FIG. 3F shows ECL intensity curve of the various concentration of the Hp: (a) 0 pg mL⁻¹, (b) 0.1 pg mL⁻¹ (c) 1 pg mL⁻¹, (d) 10 pg mL⁻¹, (e) 100 pg mL⁻¹, (f) 1 ng mL⁻¹ and (g) 10 ng mL⁻¹, in accordance with the illustrative embodiment of the present invention.

FIG. 4 shows a comparison of different immunosensors for detection of Haptoglobin, by the illustrative embodiment.

FIG. 5A shows calibration plot of the Hp-immunosensors in detecting Hp recorded for 0.1 fg mL⁻¹ to 10 ng mL⁻¹ Hp;

FIG. 5B shows reproducibility, where panel (a-e) shows the pairs of the Hp-immunosensor without Hp (0 ng mL⁻¹) and with Hp (1 ng mL⁻¹) showing a consistent and same ECL signal;

FIG. 5C shows long-term stability of ECL Hp-immunosensor studied for 28 days: where panel (a-e) shows the pair of the bars with ECL peak (a) without Hp (0 ng mL⁻¹) and with Hp (1 ng mL⁻¹) on zero-day, (b) without Hp (0 ng mL⁻¹) and with Hp (1 ng mL⁻¹) on 7^{th}day, (c) without Hp (0 ng mL⁻¹) and with Hp (1 ng mL⁻¹) on 14^{th}day, (d) without Hp (0 ng mL⁻¹) and with Hp (1 ng mL⁻¹) on 2^{st}day, and (e) without Hp (0 ng mL⁻¹) and with Hp (1 ng mL⁻¹) on 28^{th}day;

FIG. 5D shows Interference-resistant of ECL Hp-immunosensors, where (a) 0 pg mL⁻¹ Hp, (b) 100 pg mL⁻¹ Hp, and from bar (c to o) shows the ECL intensity of 100 pg mL⁻¹ Hp mixed with 1000 pg mL⁻¹: (c) CRP, (d) Cortisol, (e) DHEA, (f) AFP, (g) Leptin, (h) AA, (i) BSA, (j) CEA, (k) UA, (l) hCG, (m) β2m, (n) IgA and (o) cocktail of all antigens;

FIG. 5E shows Selectivity of ECL Hp-immunosensors, where (a) 0 pg mL⁻¹, (b) 100 pg mL⁻¹ Hp, and from bar (c to o) shows the ECL signal of 0 pg mL⁻¹ Hp mixed with 100 pg mL⁻¹; (c) CRP, (d) Cortisol, (e) DHEA, (f) AFP, (g) Leptin, (h) AA, (i) BSA, (j) CEA, (k) UA, (l) hCG, (m) β2m, (n) IgA and (o) mixture of all antigens; and

FIG. 5F shows real serum sample analysis, by an illustrative embodiment of the present invention.

### DETAILED DESCRIPTION OF THE PREFERRED EMBODIMENTS

Reference will now be made in detail to the preferred embodiments of the present invention, examples of which are illustrated in the accompanying drawings.

In the following detailed description, numerous specific details are outlined to provide a thorough understanding of the invention. However, it will be understood by those of ordinary skill in the art that the invention may be practiced without these specific details. In other instances, well-known methods, procedures and/or components have not been described in detail so as not to obscure the invention.

The embodiment will be more clearly understood from the following description of the methods thereof, given by way of example only concerning the accompanying drawings.

As discussed hereinabove, Haptoglobin (Hp) can be a useful biomarker for diagnosing various diseases such as carcinoma, tissue necrosis, coronary artery, schizophrenia, diabetes mellitus, renal disease and endocrine imbalance, intravascular hemolysis, anemia, malaria, liver disease, jaundice, cirrhosis, mononucleosis and transfusion of incompatible blood and in the event of an inflammatory stimulus such as infection, injury or malignancy, whether local (vascular) or systemic (extravascular), modulating the immune response, autoimmune diseases, and significant inflammatory disorders. Hence, it is essential to fabricate an immunosensor for use in rapid point-of-care detection and quantification of Hp in serum.

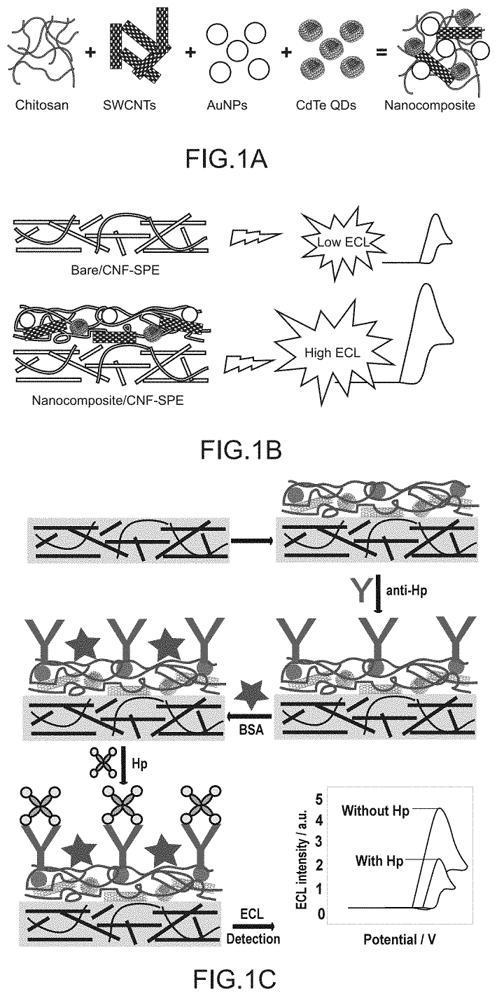

In some embodiments, the present invention discloses an electrochemiluminescence immunosensor for detecting Haptoglobin (Hp) in biological samples. The immunosensor includes nanocomposite of gold nanoparticles, single-walled carbon nanotubes, quantum dots, and chitosan, as shown in FIG. 1A. In some embodiments, the immunosensor is label-free. In some embodiments, the quantum dots are based on, for example, cadmium telluride.

The screen-printed electrode (SPE) has been widely adopted when using a three-electrode system to fabricate analytical tools due to its various advantages, including its low-cost and straightforward production protocol, with potential for mass production and miniaturization. Besides, SPEs are highly sensitive, and analysis can be performed quickly (Ahmed et al., 2015). Since ECL behavior depends on electrode materials, therefore the present invention involves the nanocomposite forming a thin film with carbon nanofibers screen-printed electrode (CNFs-SP) by modifying thereof, by an embodiment.

In some embodiments, the present invention involves [Ru(bpy)₃]²⁺ as a luminophore on the interface of the CNFs-SPE and nanocomposite modified CNFs-SPE. Also, the Tripropylamine (TPrA) is a coreactant on the interface of the CNFs-SPE and nanocomposite modified CNFs-SPE. When the ECL intensity of [Ru(bpy)₃]²⁺/TPrA system is compared with CNFs-SPE and CdTe/AuNPs/SWCNTs/CS/CNFs-SPE, ˜200% high ECL intensity is observed with CdTe/AuNPs/SWCNTs/CS/CNFs-SPE in comparison to CNFs-SPE, as shown in FIG. 1B. Also, CdTe/AuNPs/SWCNTs/CS/CNFs electrode shows 200% increase in heterogeneous electron transfer rate constant (k) and ˜200% increase in effective surface as compared to the CNFs-SPE. The immunosensor is configured to detect Hp in any biological samples, such as including but are not limited to blood, plasma, and so on.

Hence, the fabricated CdTe-QDs/AuNPs/SWCNTs/CS/CNFs-SPE immunosensor has advantages of high effective surface area, electronic conductivity, highly sensitive and selective, enabling label-free detection of Hp. The immunosensor is configured to detect Hp with a detection limit of 100 fg mL⁻¹ and has a dynamic range of 0.1 pg mL⁻¹ to 10 ng mL⁻¹. The immunosensor also exhibits 2×10⁴ fold increased sensitivity and high selectivity in the presence of most common 12 different non-target proteins and biomolecules{C-reactive protein (CRP), cortisol, dehydroepiandrosterone (DHEA), alpha fetoprotein (AFP), leptin, ascorbic acid (AA), bovine serum albumin (BSA), uric acid, beta-2-microglobulin (β2M), human chorionic gonadotropin (hCG) carcinoembryonic antigen (CEA)}found in the human serum. In some embodiments, resistant to interfere in presence 10-times higher concentration of most common 12 different non-target proteins and biomolecules found in the human serum.

The immunosensor is configured to show high stability and efficiency up to one month when the immunosensor is stored at 4° C. and studied at the interval of one week. The immunosensor can detect Hp in real serum sample as 2.5-3.1% RSD is obtained when 1, 10 and 100 ng mL⁻¹ of Hp is studied. The immunosensor requires very less fabrication time, and also the time of forming the immunocomplex thereof with anti-Hp immunocomplex is only 30 min, and analysis is just in a few seconds. The immunosensor allows direct measurement of a target without the use of an enzyme or the need for pretreatment or labeling of the target. The immunosensor requires less concentrated and smaller volumes of luminophore and co-reactant in an eco-friendly non-toxic and non-hazardous solvent. It has a potential for mass production, and penitential to be stored for one month and distant shipping at 4-8° C.

### Experimental Methods

10 μL of CdTe-QDs/AuNPs/SWCNTs/CS-nanocomposite was drop-casted onto CNFs-SPE and left to dry at RT for 1 hour to allow for the formation of a thin film of a nanocomposite. The surface was subsequently washed with water to remove unbound CdTe-QDs/AuNPs/SWCNTs/CS-nanocomposite and dried under N₂, followed by the spiking of 50 μg mL⁻¹ anti-HP (10 μL) onto the CdTe-QDs/AuNPs/SWCNTs/CS/CNFs-SPE and incubation for three h at 37° C. The electrode underwent washing with PBS and drying under N₂. Subsequent incubation with 1% BSA in 0.1% NaN₃ (w/v) for 90 min at 37° C. was performed to reduce nonspecific binding sites as shown in FIG. 1C.

After washing and drying, the sensor was stored at 7° C. until used. The electrochemical change and surface topography for each step of the immunosensor fabrication was studied by ECL, cyclic voltammetry and field emission scanning electron microscope (FE-SEM), respectively. At this stage, this Hp-immunosensor can be represented as BSA/anti-HP/CdTeQDs/AuNPs/SWCNTs/CS/CNFs-SPE.

For detection of Hp using the immunosensor, a series of Hp solutions of varying concentration (10 μL) were spiked on BSA/anti-HP/CdTe-QDs/AuNPs/SWCNTs/CS/CNFs-SPE and incubated for 30 min at 37° C. to allow for the formation of the immunocomplex. This was followed by a wash step using PBS and a drying step using N₂. ECL detection of the immunosensor was performed with 1 mL [Ru(bpy)₃]²⁺and 1 mL TPrA in 1:100 molar ratio and the cell volume maintained at 4 mL by the addition of PBS. All ECL signals were measured at an initial voltage of 0.2 V, a high voltage of 1.25 V, a low voltage of 0.2 V, a scan rate of 100 mVs⁻¹, amplifying series of 2, sensitivity (A/V) of 1.e⁻⁰⁰² and PMT voltage of 700 V.

### Results

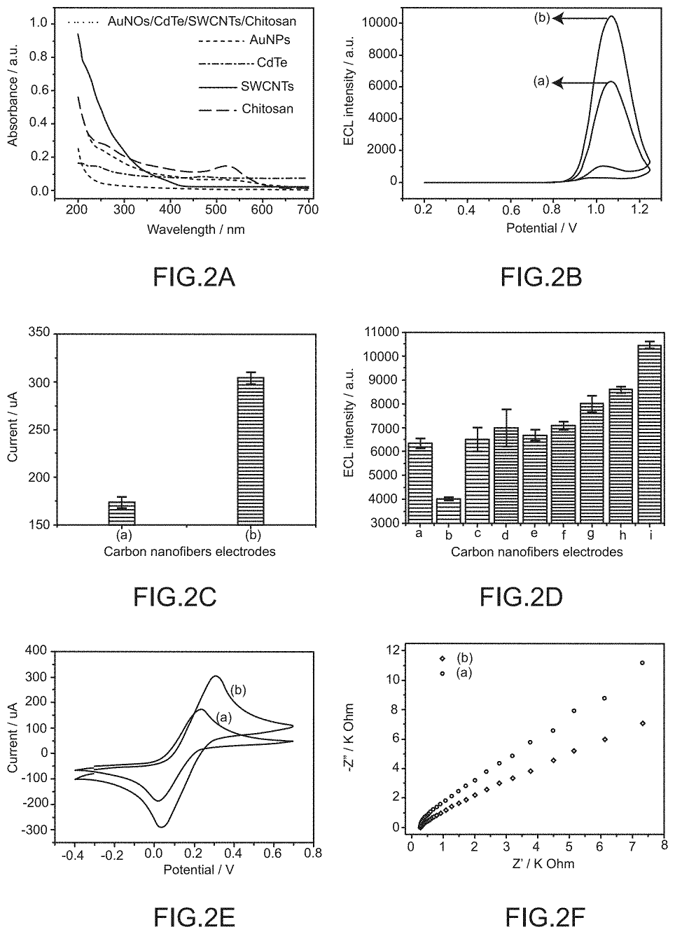

The synthesized CdTe-QDs/AuNPs/SWCNTs/CS-nanocomposite was analyzed using a nanophotometer. AuNPs is known to show an absorption peak (FIG. 2A) at 520-530 nm and with the absorption peak exhibited by the nanocomposite located at 520-530 nm shows the complete formation of the CdTe-QDs/AuNPs/SWCNTs/CS nanocomposite.

The ECL intensity curve of the [Ru(bpy)₃]²⁺/TPrA with the bare CNFs-SPE and CdTe-QDs/AuNPs/SWCNTs/CS-modified CNFs-SPE is shown in the FIG. 2B curve (a) and FIG. 2B curve (b) and shown the difference by bar diagram in FIG. 2C bar (a) and FIG. 2C bar (b) respectively. Further, [Ru(bpy)₃]²⁺/TPrA was studied with bare CNFs-SPE, CS-, SWCNTs/CS-, CdTe-QDs/CS, AuNPs/CS-, CdTe-QDs/SWCNTs/CS-, AuNPs/SWCNTs/CS-, CdTe-QDs/AuNPs/CS-, and CdTe-QDs/AuNPs/SWCNTs/CS-modified CNFs-SPE as shown respectively in FIG. 2D from bar (a-i). The CdTe-QDs/AuNPs/SWCNTs/CS-modified CNFs-SPE give highest ECL intensity. This enhanced ECL intensity is ˜200% more than the bare CNFs-SPE due to the changing electrode and electrode materials changing the ECL intensity.

The source of ECL intensity at the electrode surface may be due to the [Ru(bpy)₃]²⁺/TPrA and CdTe-QDs/TPrA as following reactions:

[Ru(bpy)₃]^{2±}→[Ru(bpy)₃]³⁺+e⁻

[Ru(bpy)₃]³⁺+TPrA→[Ru(bpy)₃]²⁺+TPrA·

TPrA→TPrA·+e⁻

[Ru(bpy)₃]³⁺+TPrA·→[Ru(bpy)₃]²*

[Ru(bpy)₃]²*→[Ru(bpy)₃]²⁺+hv

TPrA−e−→TPrA⁺·

TPrA+·−H⁺→TPrA·

TPrA·+CdTe→CdTe⁻·

TPrA+·+CdTe⁻·→CdTe*

CdTe−e→CdTe⁺·

CdTe⁺·+CdTe⁻·→CdTe*

CdTe*→CdTe+hv

Current-Potential (CV) was used to compare the electronic current of the bare CNFs-SPE and the CdTeQDs/AuNPs/SWCNTs/CNFs-SPE as shown in the FIG. 2E(a) and FIG. 2E(b) respectively. Bare CNFs-SPE demonstrated lesser electronic conductivity in comparison to the CdTeQDs/AuNPs/SWCNTs/CNFs-SPE. This may be due to the highly conductive AuNPs, SWCNTs, CdTe-QDs, and CNFs-SPE. Further, the % increase in the surface area of the CdTeQDs/AuNPs/SWCNTs/CNFs-SPE in comparison of the bare CNFs-SPE was calculated using the Randles-Sevcik Equation (i)

ip=2.69×10⁵n^{3/2}AD^{1/2}V^{1/2}Co (i)

where, ip is the peak current (A), n is the number of electrons, A=effective electrode area, D=diffusion coefficient 6.7×10⁻⁶(cm²s⁻¹), V is the scan rate (Vs⁻¹) and Co=concentration (mol cm⁻³).

From the above equation, the electrode effective surface area A for the bare CNFs-SPE and QDs/AuNPs/SWCNTs/CNFs-SPE can be determined. Hence, CdTeQDs/AuNPs/SWCNTs/CNFs-SPE possessed ˜200% more effective surface area than the bare CNFs-SPE as well as showed 200% more electronic conductivity due to synergetic electron conducting behavior of the AuNPs, CdTe-QDs, and SWCNTs on the CNFs and high surface area of the nanomaterials.

The CV was performed with [Fe(CN)₆]^{3/4-} redox probe containing 0.1 M KCl in solution from −0.4 to 0.7 V with a scan rate of 100 mVs⁻¹. Further, the electrochemical behavior of the CdTe-QDs/AuNPs/SWCNTs/CS/CNFs-SPE was investigated by electrochemical impedance spectroscopy (EIS) using redox mediator [Fe(CN)₆]^{3/4-}as shown in the FIG. 2F. The plots are composed of semi-circles and straight-line portions, representing charge transfer (Rct) and diffusion-controlled processes, respectively. In Nyquist plots, the electron transfer resistance (Rct) on electrodes surface is related to the diameter of a semi-circle. As shown in FIG. 2F, the electron transfer resistance (Rct) of the bare CNFs-SPE FIG. 2F(a) is around half of the CdTe-QDs/AuNPs/SWCNTs/CS/CNFs-SPE FIG. 2F(b). The lower Rct value of the CdTe-QDs/AuNPs/SWCNTs/CS/CNFs-SPE attributes to the lower charge transfer resistance and corresponding higher electrochemical kinetics at the surface of the modified electrode. This result was also confirmed by calculating k (the heterogeneous electron transfer rate constant) for the bare CNF-SPEs and CdTe-QDs/AuNPs/SWCNTs/CS/CNFs-SPE by using the Equation (ii)

k=RT/[(nF)2C₀ARct] (ii)

It was found that k_{Bare}=63.8 10⁻⁹ cms⁻¹ and k_{Modified}=133.7 cms⁻¹, reflecting that the CdTe-QDs/AuNPs/SWCNTs/CS/CNFs-SPE is kinetically 200% more favorable for electron conduction. Where “Rct ” is the charge transfer resistance, “T” is the temperature, “R” is the gas constant, “F” is the Faraday constant, “Co” is the concentration of a solution and “A” is the effective area of the electrode. ESI was performed with [Fe(CN)₆]^{3/4-} between 100 kHz and 0.1 Hz with a frequency 50 Hz ac amplitude of 5 mV rms.

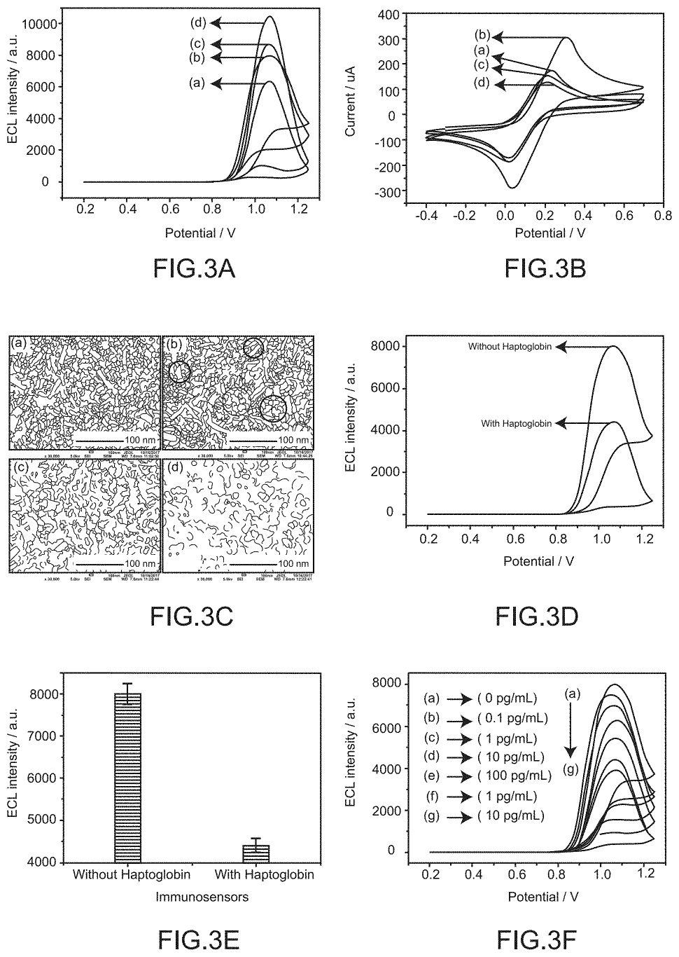

ECL technique was used for each step of the layer-by-layer fabrication of the Hp-immunosensors using [Ru(bpy)₃]²⁺/TPrA during each modification: bare CNFs-SPE, CdTe-QDs/AuNPs/SWCNTs/CS/CNFs-SPE, anti-Hp/CdTeQDs/AuNPs/SWCNTs/CS/CNFs-SPE, and BSA/ant-Hp/CdTe-QDs/AuNPs/SWCNTs/CS/CNFsSPE as shown in FIG. 3A. It shows modification of CNFs-SPE with CdTeQDs/AuNPs/SWCNTs/CS produced an increased ECL intensity (FIG. 3A(d)) compared to bare CNFs-SPE (FIG. 2A(c)) due to ECL intensity produced by both [Ru(bpy)₃]²⁺/TPrA and CdTeQDs/TPrA system on highly conductive CdTe-QDs/AuNPs/SWCNTs/CS/CNFs-SPE having high quantum efficiencies due to CdTe-QDs. Addition of anti-Hp reduced ECL intensity due to steric hindrance and formation of the insulating layer by the anti-Hp (FIG. 3A(b)) which reduced the diffusion of the [Ru(byp)₃]Cl₂-TPrA on the electrode surface. Incubation with BSA further reduced ECL signal, confirming the successful adsorption of BSA as a blocking agent for nonspecific binding (FIG. 3A(a)) and formation of insulation layer by the BSA.

Further characterization of the layer-by-layer fabrication of the immunosensor was performed using cyclic voltammetry as shown in FIG. 3B with well-defined anodic and cathodic peak currents corresponding to the oxidation and reduction of the Fe(CN)₆]^{3/4-} a mediator. A similar trend of anodic and cathodic peak currents with Fe(CN)₆]^{3/4-} was observed at each fabrication stage and reduction in the current due to the formation of the insulating layers with the ECL intensity characterization of the immunosensors.

Besides, each step of fabrication was studied using FE-SEM in situ (FIG. 3C(a-d)). In FIG. 3C(a), the fibrous structures on the surface of bare-CNFs-SPE denoted the carbon nanofibers. The change in surface topography was evident upon the addition of the CdTeQDs/AuNPs/SWCNTs/CS-nanocomposite on CNFs-SPE FIG. 3C(b), showing the presence of the spherical AuNPs, CdTe-QDs, and tubular SWCNTs. Complete immobilization of anti-Hp on CdTe-QDs/AuNPs/SWCNTs/CS/CNFs-SPE was indicated by the porous surface with a coarse appearance as shown in FIG. 3C(c), which may be attributed to the globular nature of anti-Hp. Upon incubation with BSA, the surface became smoother with filled pores at the surface as nonspecific binding surfaces were blocked FIG. 3C(d).

Further, the surface charge of the immunosensor was compared in the presence of the positively and negatively charged probe using Chourocuolometry (CC). The label-free ECL immunosensor (BSA/anti-Hp/CdTeQDs/AuNPs/SWCNTs/CS/CNFs-SPE) with [Ru(byp)₃]Cl₂-TPrA system was preliminary investigated to detect signal without Hp (0 ng mL⁻¹) and with Hp (1 ng mL⁻¹) FIG. 3D, a comparison is shown in FIG. 3E with bar diagram. The immunosensor produced a lower signal with Hp and higher signal without Hp. This is due to the formation of antigen-antibody (anti-Hp-Hp) immunocomplex reduced the diffusion of the [Ru(byp)₃]Cl₂-TPrA toward the electrode surface due to the steric hindrance.

CC study inferred that the Hp-immunosensor bear slight positive charge in the presence the Hp, which also contribute in weakly repelling the diffusion of the [Ru(byp)₃]²⁺ on the electrode surface by the electrostatic repulsive interaction and reduce the ECL intensity. This quantitative Hp concentration-dependent reduction in the ECL signal can be tracked for the low Hp detection ability of the immunosensor to the maximum concentration (saturation level of the immunosensor) detection ability. Therefore, different concentrations of Hp (0.1 pg mL⁻¹ to 10 ng mL⁻¹) were used to examine the ECL intensity.

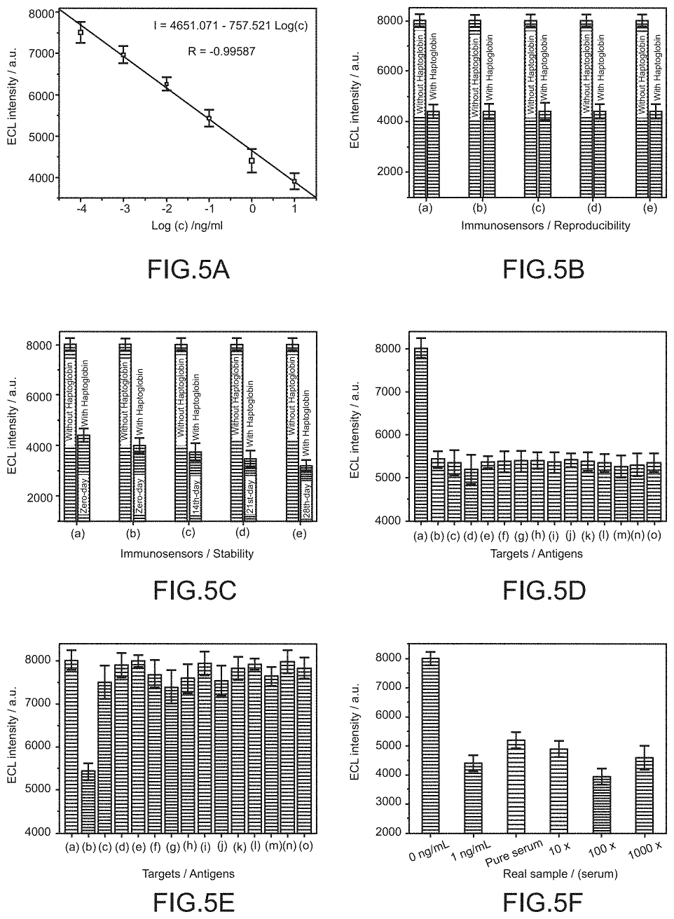

FIG. 3F shows experimental ECL intensity dose-response curve for the fabricated immunosensor in the presence of (a) 0 pg mL⁻¹, (b) 0.1 pg mL⁻¹, (c) 1 pg mL⁻¹, (d) 10 pg mL⁻¹, (e) 100 pg mL⁻¹, (f) 1 ng mL⁻¹ and (g) 10 ng mL⁻¹reflecting increase in concentration of Hp, the signal decreases and reached to saturation due to thicker antigen-antibody immunocomplex formation and large steric-hindrance which reduces the diffusion of the [Ru(bpy)₃]Cl₂-TPrA toward the electrode surface. Further repulsive electrostatic interaction between [Ru(bpy)₃]²⁺ and immunosensor with Hp also reduces the ECL intensity. The calibration plot was plotted displaying a negative linear relationship between log[c] of Hp and ECL intensity from 0.1 pg mL⁻¹ to 10 ng mL⁻¹ (R=0.99587) as shown in FIG. 5A. An extremely low experimentally determined LOD of 100 fg mL⁻¹ was obtained. This LOD was obtained experimentally, which is the minimum concentration of the Hp that can be truly detected in the solution. This is the minimum concentration (100 fg mL⁻¹) of Hp whose ECL intensity curve using this immunosensor can be clearly distinguished from the ECL intensity curve of the immunosensor without Hp (0 fg mL⁻¹). This low LOD may be partly due to the highly conductive CNFs-SPE modified with the CdTe-QDs/SWCNTs/CS-nanocomposite of various nanomaterials, with ˜200% higher ECL intensity, ˜200% higher effective surface area and 200% higher electron conduction. Moreover, the CNFs in the electrode surface facilitates rapid electron transfer and contribute to high ECL signal due to the presence of more edge plane defects and the availability of the larger surface area of the carbon nanofibers.

ECL intensity gets further increased due to the presence of the SWCNTs which increases the electronic signal due to the larger surface at the same site than the surface of a bare electrode due to high aspect ratio, AuNPs with the splendid conductivity, large surface area, high biocompatibility and antibody immobilization properties, excellent biocompatibility and high quantum efficiency of the CdTe-QDs as well as additional luminophore at the surface of the electrode and; highly biocompatible CS with a property to enhance ECL which collectively contribute in enhancing the ECL intensity at the electrode surface in the presence of the [Ru(bpy)₃]Cl₂-TPrA and enhance sensitivity of the immunosensor for the detection of the low concentration (100 fg mL⁻¹) of the Hp. The biocompatibility and high aspect ratio of the SWCNTs, biocompatibility of the CdTe-QDs assist in binding large anti-Hp via Vander Waals forces; property of AuNPs to interact the antibodies through electron transition complexes and property of CS to interact with the antibody through electrostatic interaction can assist in binding large amounts of anti-Hp which may form immunocomplex even in the presence of minute Hp concentration.

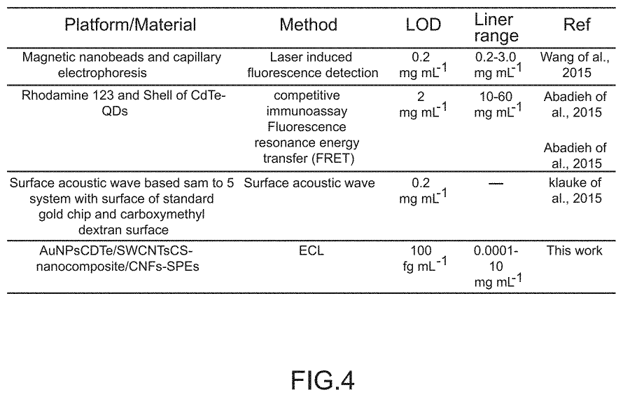

ECL is having high sensitivity and versatility, low background signal, simple optical setup and provide reasonable temporal and spatial control, produces decreased ECL signal even for low concentration of the Hp on highly conductive CdTe-QDs/AuNPs/SWCNTs/CS-nanocomposite based immunosensor and enhance sensitivity. On the other hand [Ru(bpy)₃]Cl₂-TPrA and CdTe-QDs-TPrA produce high ECL in the presence of high applied voltage and detect a minute concentration of the Hp (100 fg mL⁻¹). Hence, such a highly sensitive label-free Hp-immunosensor with a wide dynamic range of detection can be used to detect and monitor minute concentration (100 fg mL⁻¹) of the Hp in real serum; when the Hp level decline in case of diseases such as anemia, jaundice and cirrhosis, while it can also be used to detect and monitor the Hp concentration in the serum when the Hp level rises much fold in diseases such as carcinoma, coronary artery and schizophrenia. A comparison of the performance of the fabricated label-free ECL Hp-immunosensor versus available immunosensors is shown in FIG. 4.

The reproducibility responses of five fabricated Hp-immunosensor was evaluated (FIG. 5B), which demonstrated a consistent high ECL signal. For stability study, immunosensors were fabricated and kept in a refrigerator from 0 to 28 days at 4° C. and tested with one ng mL⁻¹ of Hp at an interval of one week as shown in FIG. 5C. As the number of days increased, the ECL intensity response steadily decreased, and it was found 73% on the 28th day.

A further interference study was performed to study the performance of the Hp immunosensor in the presence of the most common non-target antigen found in the serum (FIG. 5D). It shows that Hp (100 pg mL⁻¹) mixed with ten times higher in concentration with of each interference (non-target antigens=1000 pg mL⁻¹) or cocktail of all the antigen, display the similar ECL signal. A selectivity study was carried out to compare selectivity performance (FIG. 5E) with non-target antigens. ECL Hp-immunosensor demonstrated high selectivity towards Hp. Therefore, it validates that the fabricated immunosensor is highly interference-resistant and selective.

The feasibility of the label-free ECL Hp-immunosensor for the practical application was investigated by examining the ability of the sensor in detecting Hp in human serum. A serum sample was diluted with PBS buffer 10×, 100× and 1000× after that 1 ng mL⁻¹ Hp was added to each dilution including in 1× (pure undiluted serum). The highest reduction in signal was detected at dilution 100× when compared to pure one ng mL⁻¹ Hp (FIG. 5F). Henceforth, two different concentrations of Hp (10 and 100 ng mL⁻¹) were subsequently analyzed for their practical application in detecting Hp in the dilution factor of 100×. The relative standard deviation (RSD) and recoveries percentage for 1, 10 and 100 ng mL⁻¹ of Hp in 100× diluted serum were in the range of 2.5-3.1% and 99-117%, respectively as shown in following Table 01. The RSD value indicated the analytical precision of the Hp-immunosensor, and it also reflected repeatability and reproducibility. The fabricated immunosensor had significant potential in detecting Hp in a real serum sample.

TABLE 01
Detection of Hp in blood serum

| | Added | Found | | |
|---|---|---|---|---|
| Dilution | conc. | conc. | % | % |
| factor | (ng/mL) | (ng/mL) | RSD | Recovery |
| 100× | 1 | 0.99 | 3.1 | 99 |
| | 10 | 11.1 | 2.8 | 111 |
| | 100 | 117 | 2.5 | 117 |

While the preferred embodiment of the present invention and its advantages have been disclosed in the above description, the invention is not limited to that but only by the scope of the appended claim.

As will be readily apparent to those skilled in the art, the present invention may readily be produced in other specific forms without departing from its essential characteristics. The present embodiments are, therefore, to be considered as merely illustrative and not restrictive, the scope of the invention being indicated by the claims rather than the preceding description, and all changes which come within therefore intended to be embraced therein.

I/We claim:

1. An electrochemiluminescence immunosensor for detecting Haptoglobin in a biological sample, the electrochemiluminescence immunosensor comprising:
a nanocomposite of gold nanoparticles;
a single-walled carbon nanotubes;
one or more quantum dots; and
a chitosan.

2. The electrochemiluminescence immunosensor of claim 1, wherein the quantum dots are based on cadmium telluride.

3. The electrochemiluminescence immunosensor of claim 1, wherein the electrochemiluminescence immunosensor is label free.

4. The electrochemiluminescence immunosensor of claim 1, wherein the nanocomposite of gold nanoparticles, single walled carbon nanotubes, quantum dots and chitosan forms a thin film with a carbon nanofibers screen-printed electrode by modifying thereof.

5. The electrochemiluminescence immunosensor of claim 1, further comprising [Ru(bpy)₃]²⁺ as a luminophore on an interface of a carbon nanofibers screen-printed electrode and a nanocomposite of gold nanoparticles, single walled carbon nanotubes, quantum dots and chitosan modified carbon nanofibers screen-printed electrode.

6. The electrochemiluminescence immunosensor of claim 5, further comprising Tripropylamine as a coreactant on the interface of the carbon nanofibers screen-printed electrode and the nanocomposite of gold nanoparticles, single walled carbon nanotubes, quantum dots and chitosanmodified carbon nanofibers screen-printed electrode.

7. The electrochemiluminescence immunosensor of claim 4, wherein the electrochemiluminescence immunosensor forms an immunocomplex on the nanocomposite of gold nanoparticles, single walled carbon nanotubes, quantum dots and chitosan modified carbon nanofibers screen-printed electrode.
